(19) United States
(12) Patent Application Publication
(10) Pub. No.: US 2022/0061882 A1
(43) Pub. Date: Mar. 3, 2022

(54) GUIDE HOLE STRUCTURES

(71) Applicant: University of Central Florida Research Foundation, Inc., Orlando, FL (US)

(72) Inventor: Sang-Eun Song, Orlando, FL (US)

(73) Assignee: University of Central Florida Research Foundation, Inc., Orlando, FL (US)

(21) Appl. No.: 17/462,402

(22) Filed: Aug. 31, 2021

Related U.S. Application Data
(60) Provisional application No. 63/072,480, filed on Aug. 31, 2020.

(51) Int. Cl. A61B 17/34

(52) U.S. Cl. CPC A61B 17/3403; A61B 2017/00911; A61B 2017/3411; A61B 17/3421

(57) ABSTRACT

A guide hole structure can include a body defining one or more guide holes through the body between a first surface and a second surface. The guide hole structure can include one or more contact area reducing features forming and/or extending from an inner surface of the body to form the one or more guide holes. The one or more contact area reducing features can be configured to reduce a contact area of a shaft inserted into and/or through the one or more guide holes.

### CROSS-REFERENCE TO RELATED APPLICATIONS

This application claims priority to and the benefit of U.S. Provisional Application No. 63/072,480, filed Aug. 31, 2020, the entire contents of which are herein incorporated by reference in their entirety.

### FIELD

This disclosure relates to guide hole structures (e.g., for shaft insertion through a guide hole).

### BACKGROUND

Existing guide hole structures, e.g., template grids used for guiding high-speed needles (e.g., for biopsy guns), change the dynamics of insertion by reducing the shaft insertion speed (e.g., needle speed) or allow for misalignment due to clearance in the holes to maintain a desired speed.

Such conventional methods and systems have generally been considered satisfactory for their intended purpose. However, there is still a need in the art for improved guide hole structures. The present disclosure provides a solution for this need.

### SUMMARY

A guide hole structure can include a body defining one or more guide holes through the body between a first surface and a second surface. The guide hole structure can include one or more contact area reducing features forming and/or extending from an inner surface of the body to form the one or more guide holes. The one or more contact area reducing features can be configured to reduce a contact area of a shaft inserted into and/or through the one or more guide holes.

In certain embodiments, the body can define a biopsy template. In certain embodiments, the one or more guide holes can include a plurality of guide holes disposed in a pattern (e.g., for the biopsy template). Any suitable pattern is contemplated herein. Any other suitable use with any suitable application (medical or otherwise) is contemplated herein.

Each guide hole can be spaced about 5 mm apart. Any other suitable spacing is contemplated herein.

In certain embodiments, the one or more contact area reducing features can include a plurality of contact area reducing features (e.g., unconnected directly and/or separately extending from the inner surface). The one or more contact area reducing features can provide a plurality of contact points, lines, and/or surfaces for the shaft to contact, for example.

In certain embodiments, the one or more contact area reducing features can include longitudinal ribs extending along an axial direction and extending radially inwardly from the inner surface of the guide hole. In certain embodiments, the one or more contact area reducing features includes lateral and/or circumferential ribs disposed along an inner circumference of the inner surface and extending radially inward from the inner surface. In certain embodiments, the one or more contact area reducing features can include a spherical embossing on the inner surface of the guide hole.

In certain embodiments, the structure can be made of metal or hard plastic (e.g., or any other suitable hard material). In certain embodiments, the structure can be made of a non-ferromagnetic material for use within an MRI.

In accordance with at least one aspect of this disclosure, a biopsy template system can include a guide hole structure as disclosed herein, e.g., as described above. Any other suitable application for any suitable embodiment of a guide hole structure is contemplated herein.

In accordance with at least one aspect of this disclosure, a method can include using a contact area reducing guide hole structure for inserting a shaft through the contact area reducing guide hole structure to reduce resistance of motion of the shaft from a contact area of the guide hole structure. In certain embodiments, inserting the shaft can include inserting a biopsy cannula through the contact area reducing guide hole structure. The method can include any other suitable method(s) and/or portion(s) thereof.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is a schematic view of an embodiment of a structure in accordance with this disclosure, showing the structure forming a template having a plurality of guide holes with a biopsy gun needle disposed therethrough;

FIG. 2A shows a cross-sectional view of an embodiment of a guide hole structure shown having a cannula disposed therein;

FIG. 2B is a perspective see-through view of the embodiment shown in FIG. 2A;

FIG. 2C shows a cross-sectional view of an embodiment of a guide hole structure shown having a cannula disposed therein;

FIG. 2D is a perspective see-through view of the embodiment shown in FIG. 2C;

FIG. 3A shows a perspective see-through view of an embodiment of a guide hole structure shown having a cannula disposed therein;

FIG. 3B is a perspective view of the embodiment shown in FIG. 3A;

FIG. 4A shows a cross-sectional view of an embodiment of a guide hole structure shown having a cannula disposed therein;

FIG. 4B is a perspective see-through view of the embodiment shown in FIG. 4A;

FIG. 5 shows a comparison in velocity of a biopsy cannula achieved through an embodiment of guide hole structure having 50% surface area contact versus an embodiment having 100% surface area contact; and

FIG. 6 shows a chart indicating the effect of friction coefficient as it relates to biopsy needle insertion speed as a function of time.

### DETAILED DESCRIPTION

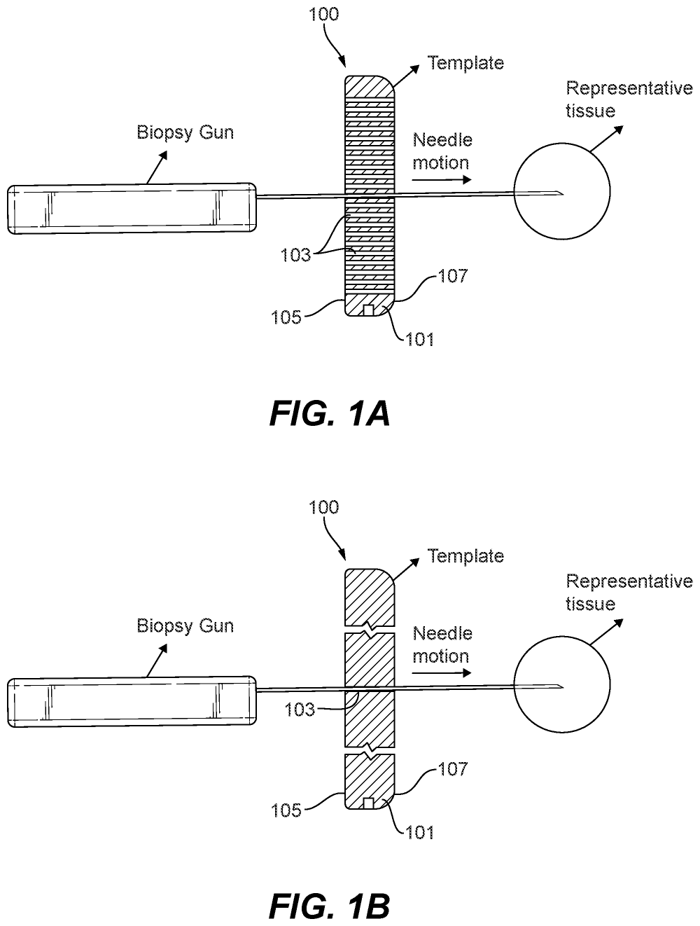

Reference will now be made to the drawings wherein like reference numerals identify similar structural features or aspects of the subject disclosure. For purposes of explanation and illustration, and not limitation, an illustrative view of an embodiment of a structure in accordance with the disclosure is shown in FIG. 1 and is designated generally by reference character 100.

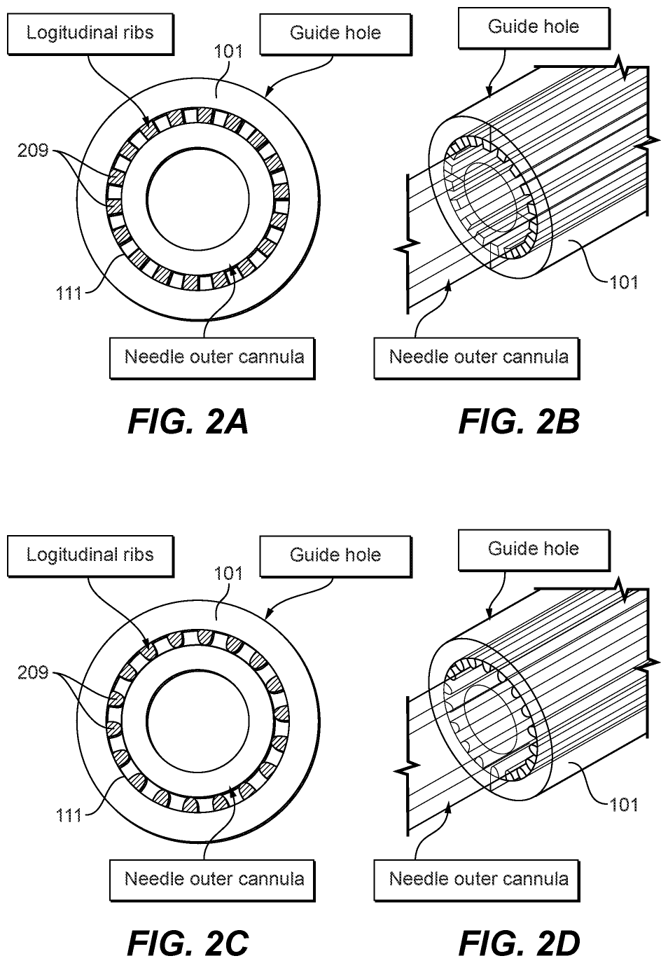

Other embodiments and/or aspects of this disclosure are shown in FIGS. 2A-6. Certain embodiments described herein can be used for biopsy needle guide holes, for example. Any suitable guided shaft insertion application is contemplated herein.

Referring to FIG. 1, a guide hole structure 100 can include a body 101 defining one or more guide holes 103 (e.g., a single guide hole, or a plurality of guide holes) through the body 101 between a first surface 105 and a second surface 107. The structure 100 can be any suitable size and have holes 103 sized to receive any suitable shaft shaped object for any suitable function (e.g., as a biopsy template for a biopsy needle).

Referring additionally to FIGS. 2A-4B, the guide hole structure 100 can include one or more contact area reducing features 209, 309, 409 (e.g., longitudinal ribs, lateral ribs, spherical embossing, etc.) forming and/or extending from an inner surface 111 of the body 101 to form the one or more guide holes 103. The one or more contact area reducing features 209, 309, 409 can be configured to reduce a contact area (e.g., relative to a cylindrical hole or other uniform cross-section passageway) of a shaft (e.g., a biopsy needle) inserted into and/or through the one or more guide holes 103.

The one or more contact area reducing features can be integrally formed with the body 101 (e.g., milled, additively manufactured, etc.), e.g., at least partially forming the inner surface 111. In certain embodiments, the one or more contact area reducing features can be attached to the inner surface 111. Any suitable method to form the body 101 and the one or more contact area reducing features 209, 309, 409 are contemplated herein.

In certain embodiments, the body 101 can define a biopsy template, e.g., as shown in FIG. 1. Any other suitable use with any suitable application (medical or otherwise) is contemplated herein. In certain embodiments, the body 101 can define a single guide hole instead of a template with a plurality of guide holes.

In certain embodiments, the one or more guide holes 103 can include a plurality of guide holes 103 disposed in a pattern (e.g., for the biopsy template). Any suitable pattern (e.g., a grid as shown) is contemplated herein. Random relative placement of a plurality guide holes 103 a minimum distance apart of holes 103 is also contemplated herein. Any suitable relative guide hole placement for any suitable purpose is contemplated herein.

Each guide hole 103 can be spaced about 5 mm apart (e.g., for biopsy template uses or any other suitable use). In certain embodiments, the guide holes 103 can be spaced apart between about 3 mm to about 10 mm. Any other suitable spacing is contemplated herein.

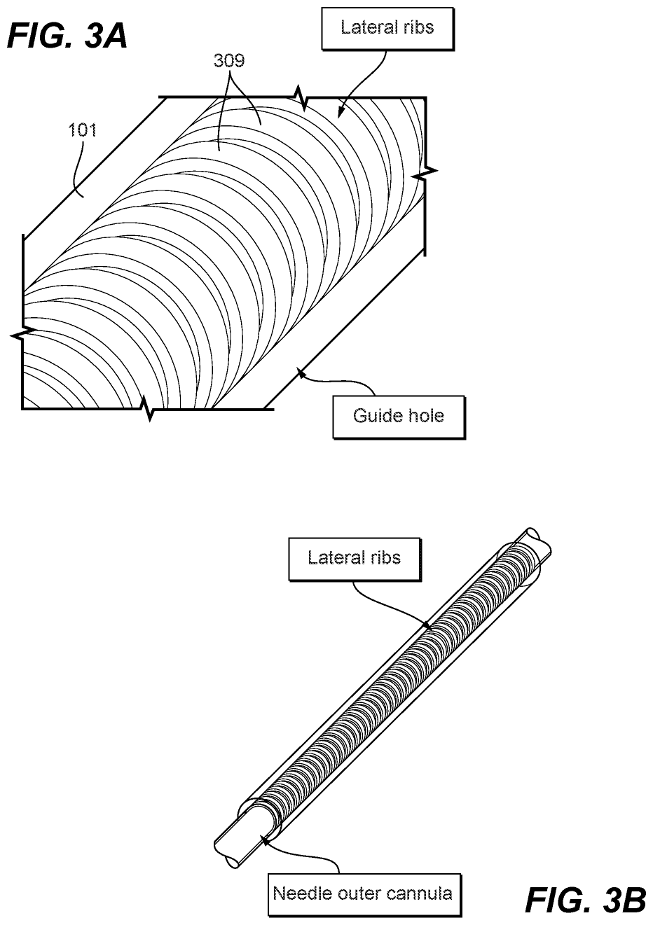

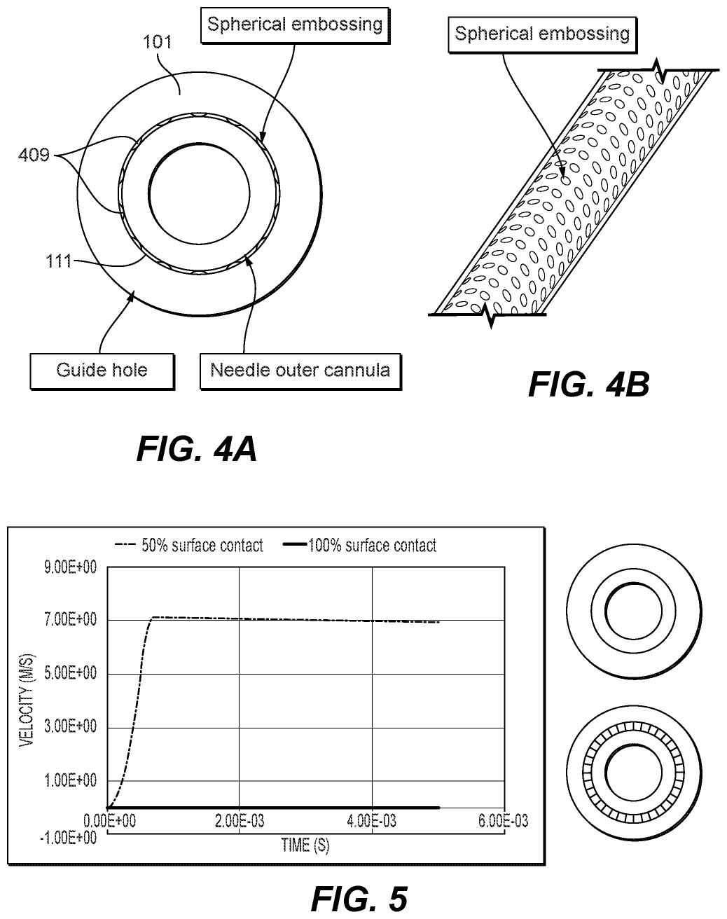

In certain embodiments, the one or more contact area reducing features 209, 309, 409 can include a plurality of contact area reducing features (e.g., unconnected directly and/or separately extending from the inner surface), e.g., as shown in FIGS. 2A-4B. The one or more contact area reducing features 209, 309, 409 can provide a plurality of contact points (e.g., as shown in FIGS. 4A and 4B), lines, and/or other surfaces (e.g., as shown in FIGS. 2A-3B) for the shaft to contact, for example.

In certain embodiments, e.g., as shown in FIGS. 2A and 2B, the one or more contact area reducing features 209 can be or include longitudinal ribs extending along an axial direction and extending radially inwardly from the inner surface 111 of the guide hole 103. The longitudinal ribs can extend the entire length of the guide hole 103, or can extend any suitable portion of the guide hole 103. Multiple sets of separated longitudinal ribs are contemplated herein. Any other suitable arrangement of one or more longitudinal ribs for the one or more contact area reducing features 209 are contemplated herein.

Any suitable shape for the longitudinal ribs are contemplated herein. For example, as shown in FIGS. 2A and 2B, the ribs can include a flat or substantially rectangular cross-section to create a surface contact. As shown in FIGS. 2C and 2D, the longitudinal ribs can include a rounded or cylindrical shape to create line contact. Any other suitable shape is contemplated herein.

In certain embodiments, e.g., as shown in FIGS. 3A and 3B, the one or more contact area reducing features 309 can include lateral and/or circumferential ribs disposed along an inner circumference of the inner surface 111 and extending radially inward from the inner surface 111. Any suitable axial thickness of the circumferential ribs is contemplated herein (e.g., about 1 mm, about 0.5 mm). Any suitable cross-sectional shape (e.g., rectangular as shown, or rounded as in FIGS. 2C and 2D) is contemplated herein. Any suitable number of circumferential ribs and/or axial spacing thereof is contemplated herein. It is contemplated that any suitable circumferential ribs can be partially circumferential.

In certain embodiments, e.g., as shown in FIGS. 4A and 4B, the one or more contact area reducing features 409 can include a spherical embossing on the inner surface 111 of the guide hole 103. The contact area reducing features 409 can include any suitable semisphere (or other suitable partial sphere shape) attached or formed on any suitable inner portion(s) of a guide hole 103. For example, the spherical embossing can be located along the entire axial length of the guide hole 103, or in any suitable patches or locations. Such embodiments can create point contact with the needle cannula. Any suitable size spherical shapes (all being the same or any suitable amount having one or more different sizes), and any suitable pattern, spacing, or randomize location is contemplated herein. Any other suitable shape for the embossing (e.g., non-spherical) is contemplated herein.

While certain embodiments of contact area reducing features are shown, any other suitable shapes, numbers of shapes and/or features, locations, and/or patterns within some and/or each guide hole 103 is contemplated herein.

In certain embodiments, the structure 100 can be made of metal or hard plastic (e.g., or any other suitable hard material). The structure 100 can be made using any suitable method (e.g., molding, machining, additive manufacturing, etc.). In certain embodiments, the structure can be made of a non-ferromagnetic material (e.g., aluminum, titanium) for use within an MRI machine.

In accordance with at least one aspect of this disclosure, a biopsy template system can include a guide hole structure as disclosed herein, e.g., as described above. Any other suitable application for any suitable embodiment of a guide hole structure is contemplated herein.

In accordance with at least one aspect of this disclosure, a method can include using a contact area reducing guide hole structure for inserting a shaft through the contact area reducing guide hole structure to reduce resistance of motion of the shaft from a contact area of the guide hole structure. In certain embodiments, inserting the shaft can include inserting a biopsy cannula through the contact area reducing guide hole structure. The method can include any other suitable method(s) and/or portion(s) thereof. Embodiments can be used for any suitable procedure, e.g., a percutaneous medical procedure.

Percutaneous intervention procedures are employed in numerous surgical methods involving needle puncture e.g., core needle biopsy, brachytherapy, cryotherapy (e.g., cryoablation), etc. for soft tissues like prostate, breast, liver etc. Core needle biopsy (CNB) is a typical biopsy procedure in which a hollow needle is used for extracting tissue samples from an organ in order to examine it for any possible malignancies. The sample is cylindrical in shape and referred to as a core. CNB is usually performed under the guidance of imaging, e.g., Ultrasound (US), Computed Tomography (CT) or Magnetic resonance Imaging (MRI).

Irrespective of the imaging modality, the commonly used tissue sampling device includes a fully-automatic or semi-automatic biopsy gun. The biopsy gun is loaded with a biopsy needle that cuts through the tissue and collects the sample for further examination. Biopsy needles have an inner and an outer cannula. The inner cannula consists of a notch that collects the sample. The difference between the fully-automated and semi-automated gun lies in the way the inner cannula of the needle is positioned. Both the guns use a spring-loaded trigger system. In a fully-automated system two-stage spring action is employed. During biopsy, as the gun is fired, in the first stage the spring force pushes the inner cannula followed by the second stage where outer cannula is pushed that cuts through the tissue. The spring motion is initiated by cocking the gun followed by first and second trigger for inner and outer cannula, respectively. In contrast, for a semi-automated gun, the inner cannula is positioned manually followed by quick spring-loaded triggering of the outer cannula for cutting.

Several of these CNB procedures can utilize a template guide (a grid of guide holes) for physically guiding a biopsy needle to the target location. Such a guide enables accurate and precise targeting of the tumor or other biopsy target. Targeting can be affected by certain factors such as hand tremor during biopsy gun handling, the physical guide (e.g., the template grid), and the interaction between the needle and the tissue. The first factor has been addressed in the past by several researchers by development of robotic systems that eliminate the variability due to human factors. The third factor has been addressed in some cases by development of mathematical models that consider the deflection of needle due to tissue interaction and adjust the trajectory accordingly. The second factor which includes the influence of template guidance (guide holes) on tissue biopsy has been largely ignored in the art. While certain studies have shown that slow speed of insertion leads to lower deflection of needle and better accuracy, the results are not be applicable to a clinical scenario since a high speed of insertion is a basic requirement for tissue biopsy using a biopsy gun.

Certain studies have shown that dynamic behavior of the biopsy needle that includes cutting speed can have significant effect on the success of biopsy and high speeds were recommended for a successful biopsy of a heterogeneous tissue. It has been determined herein that the influence of the guide hole (template grid) on the dynamics of needle insertion can play a significant role in determining the quality of tissue biopsy, i.e., interaction of needle and guide hole can play a significant role in determining the quality of tissue biopsy.

At present the typical template guides used have an array of straight holes drilled at 5 mm distance to each other. The hole diameter depends on the biopsy needle size, e.g., 14 gauge, 16 gauge, or 18 gauge needles. Enough clearance needs to exist to enable high speed needle motion while it should be tight enough to enable accurate targeting (or the guide hole will serve no purpose). As a result of these opposing factors at play, in practice, biopsy gun speeds are compromised and this may lead to low quality samples. Accordingly, embodiments include a guide hole design that enables accurate and precise needle targeting while minimizing the change in dynamic characteristics enabling the biopsy needle firing at the designed speed leading to a successful intervention.

FIG. 1 shows an embodiment of a structure (e.g., a template grid) having guide holes 103 for a guided needle intervention procedure. A biopsy gun is shown being directed through a guide hole to align the needle and target a biopsy tissue (e.g., a tumor). In traditional templates and guide holes, when the biopsy gun is being handled by the physician, the needle may be positioned eccentrically because of the diametric clearance between the guide hole and the outer surface of needle cannula. Traditionally, clearance is required to minimize contact resistance while tight fit is required for accurate and precise targeting of tumor.

FIG. 5 shows a comparison in velocity of a biopsy cannula achieved through an embodiment of guide hole structure having 50% surface area contact versus an embodiment having 100% surface area contact. As shown, FIG. 5 shows the change in needle firing speed due to different resistances of the template guide hole. In order to show the effect of reduced contact area on needle firing speed, a transient finite element analysis was performed on the embodiment shown in FIGS. 2A and 2B. Referring to FIG. 5, as shown in the plot, the reduction in contact area from 100% to 50% increases the needle shooting speed to 7.1085 m/s.

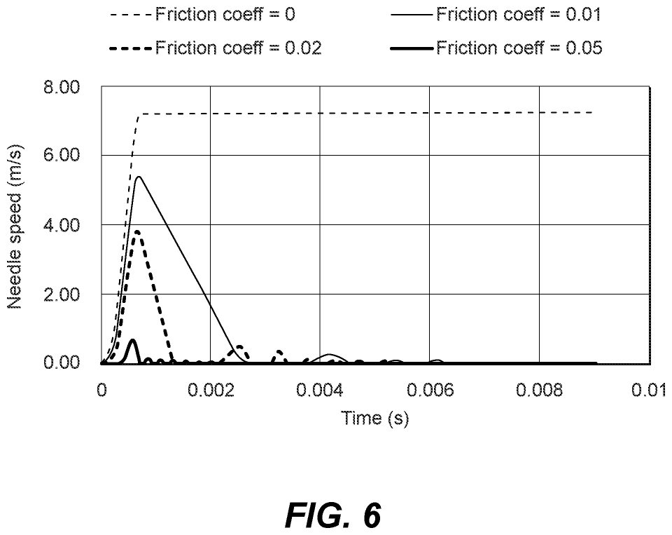

FIG. 6 shows a chart indicating the effect of friction coefficient as it relates to biopsy needle insertion speed as a function of time. FIG. 6 shows a transient structural finite element study result that was performed to simulate the biopsy needle motion through a template guide hole. The template was modeled as a single hollow cylinder with the outer surface fixed in all directions, and a needle (e.g., 18G) was modeled as a hollow cylinder to represent its outer cannula. Needle firing was simulated by applying an initial spring trigger force. The plot shows results for an ideal scenario of same inner and outer diameter for template guide hole and outer cannula of needle, respectively, with several cases representing different frictional contact properties. The result signifies the importance of contact properties on needle dynamics, e.g., the effect on speed.

Considering the procedure of needle guidance through a guide hole described above and the influence of guide hole contact properties on guidance (namely the requirement of clearance for minimum resistance and tight fight for accurate positioning for targeting, acting against each other), embodiments disclosed herein maintain the tight fit while offering minimum frictional resistance in order to deliver the biopsy or a therapy needle at the design speed.

Embodiments can provide a reduced surface contact guide hole for any application (e.g., needle insertion). Embodiments can include a template with multiple holes (e.g., each hole 5 mm apart) or a single hole. Embodiments having multiple holes can be used with images (e.g., interventional MRI) to determine which hole to use. Any suitable process is contemplated herein (e.g., interventional ultrasound, and/or any suitable navigational software). In certain embodiments, the template and/or body can be fixed to the floor via a mount, pressed against perineum, and then MRI or other navigational method can be used.

Embodiments can have any suitable shape/structure inside guide hole (e.g., rifling similar to a gun barrel shape). The contact area reducing features can be evenly spread, or can be uneven. Any suitable pattern or distribution of contact points, lines, and/or surfaces is contemplated as long as the total contact area is reduced. Certain embodiments can provide point contact surfaces.

Embodiments can include any suitable sizing for any suitable applications. For example, a biopsy needle may have a diameter of about 1 mm to about 2 mm, so the guide holes may be only slightly larger (e.g., about 1.1 mm to about 2.1 mm). Templates can have about a 10 mm or larger thickness (e.g., about 20-25 mm thickness, about 30 mm for robotic systems). Embodiments can allow reduction in thickness while maintaining accuracy because the holes can be a tighter diameter causing less diametrical play, or can allow an increased thickness while reducing contact area and friction.

Embodiments of guide hole designs as disclosed can effectively reduce the contact area between the guide hole inner diameter and a shaft (e.g., a biopsy needle). Embodiments having longitudinal ribs and/or lateral circumferential ribs, for example, the surface contact area is reduced significantly from full cylindrical contact in turn offering lesser resistance to motion. Embodiments having spherical embossing can reduce contact area to point contact. A combination of contact features can lead to linear contact in certain cases, for example.

Embodiments can enable ascertaining the highest quality specimens in case of biopsy and optimum delivery of therapy needles in procedures like brachytherapy by maintaining design speeds of the needle cannulas while being physically guided through template grids (or guide holes/guide tubes). Thus, using embodiments disclosed herein, both accurate and precise targeting and high speed needle delivery can be enabled during an intervention procedure.

Embodiments can be used for any guided needle intervention procedure such as, but not limited to, biopsy, brachytherapy, cryotherapy. Embodiments enable needle insertion at design speeds while allowing accurate and precise targeting. Due to less friction, a tight guide can be achieved for any needle insertion. Embodiments having an inner surface design with reduced contact area features can be utilized to update existing percutaneous tool guide devices e.g., prostate intervention needle template, or can be used to manufacture new templates. In certain embodiments, the guide holes can be manufactured as disposable inserts that can be inserted into reusable (e.g., sterilizable) guide devices.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. A guide hole structure, comprising:
a body defining one or more guide holes through the body between a first surface and a second surface; and
one or more contact area reducing features forming and/or extending from an inner surface of the body to form the one or more guide holes and configured to reduce a contact area of a shaft inserted into and/or through the one or more guide holes.

2. The structure of claim 1, wherein the body defines a biopsy template, wherein the one or more guide holes include a plurality of guide holes disposed in a pattern.

3. The structure of claim 2, wherein each guide hole is spaced about 5 mm apart.

4. The structure of claim 1, wherein the one or more contact area reducing features include a plurality of contact area reducing features.

5. The structure of claim 4, wherein the one or more contact area reducing features provide a plurality of contact points, lines, and/or surfaces for the shaft to contact.

6. The structure of claim 4, wherein the one or more contact area reducing features includes longitudinal ribs extending along an axial direction and extending radially inwardly from the inner surface of the guide hole.

7. The structure of claim 4, wherein the one or more contact area reducing features includes lateral and/or circumferential ribs disposed along an inner circumference of the inner surface and extending radially inward from the inner surface.

8. The structure of claim 4, wherein the one or more contact area reducing features include a spherical embossing on the inner surface of the guide hole.

9. The structure of claim 1, wherein the structure is made of metal or hard plastic.

10. The structure of claim 1, wherein the structure is made of a non-ferromagnetic material for use within an MRI.

11. A biopsy template system, comprising:
a guide hole structure, comprising:
a body defining one or more guide holes through the body between a first surface and a second surface; and
one or more contact area reducing features forming and/or extending from an inner surface of the body to form the one or more guide holes and configured to reduce a contact area of a shaft inserted into and/or through the one or more guide holes.

12. The system of claim 11, wherein the body defines a biopsy template, wherein the one or more guide holes include a plurality of guide holes disposed in a pattern.

13. The system of claim 12, wherein each guide hole is spaced about 5 mm apart.

14. The system of claim 11, wherein the one or more contact area reducing features include a plurality of contact area reducing features.

15. The system of claim 14, wherein the one or more contact area reducing features provide a plurality of contact points, lines, and/or surfaces for the shaft to contact.

16. The system of claim 14, wherein the one or more contact area reducing features includes longitudinal ribs extending along an axial direction and extending radially inwardly from the inner surface of the guide hole.

17. The system of claim 14, wherein the one or more contact area reducing features includes lateral and/or circumferential ribs disposed along an inner circumference of the inner surface and extending radially inward from the inner surface.

18. The system of claim 14, wherein the one or more contact area reducing features include a spherical embossing on the inner surface of the guide hole.

19. A method, comprising:
using a contact area reducing guide hole structure for inserting a shaft through the contact area reducing guide hole structure to reduce resistance of motion of the shaft from a contact area of the guide hole structure.

20. The method of claim 11, wherein inserting the shaft includes inserting a biopsy cannula through the contact area reducing guide hole structure.
